(19) United States
(12) Patent Application Publication
(10) Pub. No.: US 2026/0294448 A1
(43) Pub. Date: Oct. 1, 2026

(54) SYSTEM AND METHOD FOR ALIGNING A CUT GUIDE ON A BONE

(71) Applicant: Think Surgical, Inc., Fremont, CA (US)

(72) Inventors: Jonathan Gibson, Fremont, CA (US); Joel Zuhars, Fremont, CA (US)

(73) Assignee: Think Surgical, Inc., Fremont, CA (US)

(21) Appl. No.: 19/577,529

(22) Filed: Mar. 25, 2026

Related U.S. Application Data
(60) Provisional application No. 63/779,564, filed on Mar. 28, 2025.

(51) Int. Cl. A61B 17/17; A61B 17/00; A61B 34/20; A61B 34/30

(52) U.S. Cl. CPC A61B 17/1764; A61B 34/20; A61B 34/30; A61B 2017/0046; A61B 2034/2055; A61B 2034/302

(57) ABSTRACT

A guide is provided that includes a first guide tube having a first bore sized to accommodate a first bone pin and a bone contacting surface configured to contact a bone surface. A second guide tube having a second bore sized to accommodate a second bone pin or a surgical boring tool is also provided. A cross-bar portion interconnects the first guide tube and the second guide tube and is configured to space the first bore a pre-determined distance from the second bore. A process for aligning a cut guide on a bone at a pre-determined location using the guide is also provided. A system is also provided that includes the guide, a robotic device, and a computing system operative for forming holes in the bone for receiving a portion of a cut guide therein for aligning the cut guide on the bone at a pre-determined location.

### RELATED APPLICATIONS

This application claims priority benefit of U.S. Provisional Application Ser. No. 63/779,564 filed Mar. 28, 2025; the contents of which are hereby incorporated by reference.

### FIELD OF THE INVENTION

The present invention generally relates to computer assisted surgery, and more specifically to systems and methods that provide planar cuts in bone. The systems and methods in particular afford a more rapid formation of the femoral cuts associated with knee replacement or revision.

### BACKGROUND OF THE INVENTION

Total joint arthroplasty (TJA) is an orthopedic surgical procedure in which the worn or otherwise compromised articular surfaces of the joint are replaced with prosthetic components, or implants. A TJA procedure involving the knee joint is commonly referred to as total knee arthroplasty (TKA). TKA requires the removal of worn or damaged articular cartilage and bone in the area of the knee joint surfaces in need of being replaced. The removed cartilage and bone are then replaced with synthetic implants, typically formed of metal or plastic, to create new joint surfaces.

One of the most difficult aspects of TKA is the accurate removal of bone, referred to as bone cuts, to form cut surfaces on the remaining bone for mounting the implant thereon in a desired position and orientation (POSE). Generally, surgeons plan and make the bone cuts so the final placement of the implants when mounted onto the cut surfaces restores the mechanical axis or kinematics of the patient's leg while preserving the balance of the surrounding knee ligaments. In TKA, at least five planar bone cuts are made on the distal femur to form at least five cut surfaces, where the planar contact surfaces of a femoral implant are mounted to the cut surfaces.

Prior art FIGS. 1A-1C illustrate a patient's distal femur 10 and a contour matching femoral implant 12 for a TKA procedure, where five contact surfaces on the implant are intended to contact five cut surfaces on the femur. The anterior cut surface 14 is intended to contact the anterior contact surface 13, the anterior chamfer cut surface 16 is intended to contact the anterior chamfer contact surface 15, the distal cut surface 18 is intended to contact the distal contact surface 17, the posterior chamfer cut surface 20 is intended to contact the posterior chamfer contact surface 19, and the posterior cut surface 22 is intended to contact the posterior contact surface 21. The femoral implant 12 also includes stabilizing features in the form of pegs (23, 24) intended to be inserted into stabilizing cut features (e.g., holes, not shown) formed into the distal cut surface 18 of the femur 10. The femoral implant 12 further includes an outer articulating surface 25 that contacts the articulating surface of the tibial implant. The proximal tibia requires one planar bone cut to form a proximal cut surface, where the planar contact surface of a tibial implant is mounted to the cut surface of the proximal cut surface. Any malalignment in any one of the cut planes may have drastic consequences on the final result of the procedure and the wear pattern of the implant. The results of such misalignment might include discomfort, limited range of motion, revision, and reduced implant longevity.

A traditional TKA procedure involves the use of manual devices including the use of several cutting guides, also referred to herein as cutting blocks or jigs, to form the cut surfaces. These cutting guides typically include at least one guide slot, and various alignment and installation mechanisms to align the guide slot in a desired POSE with respect to the bone. These cutting guides require reference to various anatomical landmarks and often include the use of an intramedullary rod in order to align the guide slot in a desired POSE. Once the guide slot is aligned, a user may advance a surgical saw through the guide slot to form a cut surface. Overall, these manual tools are cumbersome, require considerable surgical experience, time intensive to deploy, and the accuracy of the placement of the cutting guides cannot be assured once mounted to the target bone, and instead, the surgeon has to trust that it is in the proper POSE.

Cutting guides, while time consuming to mount to a target bone and subsequently remove, provide notable advantages that include constraining the surgical saw to the desired POSE and helps stabilize the bone removal device during cutting to reduce deflection from the desired plane; and a single cutting guide may include multiple guide slots (referred to herein as an N-in-l cutting block) which can define more than one cutting plane to be accurately resected, such as a 4-in-l cut block, 5-in-l cut block . . . N-in-l cut block. Thus, the surgeon can resect two or more planes once the cutting guide is accurately oriented on the bone.

To overcome the tedious task of manually aligning cutting guides on the bone, several robotic surgical systems have been developed to accurately form the cut surfaces including the TS01ution One@ Surgical System (THINK Surgical, Inc., Fremont, CA) and the RIO@ Robotic Arm (Stryker-Mako, Kalamazoo, MI). The T Solution One@ Surgical System aids in the planning and execution of total hip arthroplasty (THA) and total knee arthroplasty (TKA). Other robotic systems may assist in robotically aligning a cutting guide in a desired POSE such as the ROSA@ Robotic System (Zimmer Biomet, Warsaw, IN) and the hand-held robotic surgical system described in U.S. Pat. No. 11,457,980 and incorporated herein by reference in its entirety. The hand-held robotic system includes a hand-held robotic device that robotically aligns pins with virtual planes having a pre-determined location relative to the bone. In a first step of the procedure, the pins are inserted in the bone coincident with a first virtual plane. A cut guide having one or more guide slots is then clamped onto the pins. The location of the first virtual plane, and therefore the pins inserted in the bone coincident with the virtual plane, is defined such that when the cut guide is clamped onto the pins, the guide slot is aligned with the desired POSE to form the distal cut plane. In a second step of the procedure, pins are inserted in the distal cut surface of the bone and coincident with a second virtual plane. A drill guide is then coupled to the pins inserted in the bone, where the drill guide includes a slot for receiving the pins and two guide holes spaced a defined distance apart from the slot. The guide holes are configured to receive a drill bit to guide the formation of peg holes in the distal cut surface of the bone. Pegs of a 4-in-l cut block is then mounted in the peg holes to guide the formation of the remaining cut surfaces on the femur. The location of the second virtual plane, and therefore the pins inserted in the bone coincident with the second virtual plane, is defined such that when the drill guide is coupled to the pins, the two guide holes of the drill guide are aligned to guide the formation of the peg holes in the distal cut surface at a planned location for receiving the pegs of a N-in-l cut block. As such, the guide slots of the N-in-l cut block are positioned at the planned location for forming the remaining cut surfaces on the femur.

The hand-held robotic system uses this method to assist in forming all five cut surfaces on the distal femur and the cut surface on the tibia. Nonetheless, there is a recognition that the steps between placement of the pins in the distal cut surface and the placement of the N-in-I cutting block may be improved, such as by reducing surgical time, removing hardware, or removing individual steps from the current procedure.

In addition, there are several applications where features (e.g., peg holes) or implants need to be formed or inserted in a bone (or other workpiece) at locations that are a pre-defined distance apart. For example, in spine surgery, pedicle screws need to be inserted on both sides of a vertebra by a known distance apart. In trauma surgery, screws (or fixation hardware) may need to be spaced a pre-defined distance apart in the bone for securing implants (e.g., trauma plates) on the bone. In TKA, the peg holes need to be formed a pre-defined distance apart in the distal cut surface in order to receive the pegs of the 4-in-l block. It is imperative that the spacing is correct for completing the procedure successfully. Conventional navigation systems may currently lack the accuracy and precision for accurate placement and spacing of the features or implants. For robotic systems, some robotic devices may operate (or is controllable) in less than number of degrees-of-freedom required to form these features or inserts the implants a predefined distance apart. For example, a hand-held robot that operates in two degrees-of-freedom (e.g., one translational degree-of-freedom and one rotational degree-of-freedom) may lack a controllable degree-of-freedom for accurately spacing the formation of the features or inserting the implants.

In TKA, the spacing of the peg holes is particularly important. It is imperative that the location and spacing of the peg holes formed in the bone are accurate such that the cut block is positioned on the bone at the planned location. The formation of the peg holes at the correct location, and especially the correct spacing, is particularly tedious. If the spacing is off by just a few millimeters, the pegs of the cut block cannot be mounted into the peg holes. This problem is exacerbated by the fact that the spacing between the pegs of the cut block may vary between different implant manufacturers and even different implant sizes from the same manufacturer. The spacing between the location(s) of the guide slots and the location of the pegs may also vary between different implant manufacturers and different implant sizes from the same manufacturer. Thus, there exists a need for an efficient system and method to assist in forming two or more features (e.g., peg holes), or inserting two or more implants, at accurate locations in a bone, where the two or more features are formed, or implants are inserted, at locations that are a pre-defined distance apart. There further exists a system and method for accurately aligning a cut guide in a planned (or desired) location on the bone.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1A depicts a prior art femoral bone and a femoral implant, where the femoral bone was cut to form five cut surfaces to accommodate the femoral implant.

FIG. 1B depicts a side view representation of the prior art femoral implant.

FIG. 1C depicts a perspective view representation of the prior art femoral implant.

FIG. 2 depicts prior art pins inserted in a bone coincident with a virtual plane having a predefined location with respect to the bone, where a hand-held robotic device maintains alignment of an axis of a pin coincident with the virtual plane for inserting the pins in the bone.

FIG. 3 depicts a prior art cutting guide coupled to the pins shown in FIG. 2, the cutting guide having a guide slot for guiding the formation of a distal cut surface on the femur.

FIG. 4 depicts prior art pins inserted in a distal cut surface of the bone and coincident with a second virtual plane having a predefined location with respect to the bone, where a hand-held robotic device maintains alignment of an axis of a pin coincident with the second virtual plane for inserting the pins in the distal cut surface.

FIGS. 5A and 5B depict a prior art drill guide configured to be coupled to the pins inserted in the distal cut surface.

FIG. 6 depicts the prior art drill guide of FIGS. 5A and 5B coupled to the pins inserted in the distal cut surface.

FIGS. 7A and 7B depict a prior art 4-in-l cutting block having pegs for mounting the 4-in-l cut block in peg holes formed in the distal cut surface and a plurality of guide slots for guiding the formation of a plurality of bone cuts, where prior art FIG. 7A is top perspective view thereof and prior art FIG. 7B is a bottom perspective view thereof.

FIGS. 8A and 8B depicts an inventive drill guide having guide tubes to assist in guiding the formation of peg holes in the bone, where FIG. 8A is a perspective view thereof, and FIG. 8B is a top view thereof.

FIG. 8C depicts an inventive drill guide having a first guide tube, a second guide tube, and inclusive of an adjustment feature;

FIGS. 8D-8G depict an inventive drill guide having a first guide tube, a second guide tube, and various appurtenances inclusive of a handle (FIG. 8D), a fenestration (FIG. 8E), an orthogonal shoulder (FIG. 8F) and tube base plates (FIG. 8G);

FIGS. 9A, 9B, and 10 depict a method for forming peg holes in the distal cut surface, where FIG. 9A depicts a first pin inserted in the distal cut surface and coincident with a second virtual plane having a predefined location with respect to the bone, where the first pin is inserted in the distal cut surface with the aid of a hand-held robotic device, FIG. 9B depicts the same except the first pin is inserted in the distal cut surface with the use of a drill guide and the aid of a hand-held robotic device;

FIG. 10 depicts a second pin being inserted in the distal cut surface and coincident with the second virtual plane, where the second pin is inserted in the bone with the aid of the hand-held robotic device and the drill guide of FIGS. 8A and 8B.

FIG. 11 depicts peg holes formed in the distal cut surface as a result of the removal of a first pin and a second pin from the distal cut surface.

FIG. 12 depicts a 4-in-l cut block mounted onto the distal cut surface of the femur in a planned position and orientation, where the 4-in-l cut block is aligned on the bone by inserting the pegs of the 4-in-l cut block in the peg holes formed on the distal cut surface.

FIGS. 13A and 13B depict a drill guide to assist in forming features, or inserting implants, in a bone at a pre-defined distance apart where the drill guide may be aligned relative to the bone with a single pin.

FIGS. 14 and 15 depict a procedure for forming features, or inserting implants, in a bone at a pre-defined distance apart using the drill guide shown in FIGS. 13A and 13B.

FIG. 16 depicts a surgical system to perform embodiments described herein in accordance with embodiments of the invention.

FIGS. 17A and 17B depict a hand-held robotic device to perform embodiments described herein in accordance with embodiments of the invention, where FIG. 14A depicts the robotic device in a first working POSE and FIG. 14B depicts the robotic device in a second working POSE.

### DETAILED DESCRIPTION OF THE INVENTION

The present invention has utility as a system and method for accurately forming two or more features, or inserting two or more implants, at accurate locations in a bone, where the two or more features are formed, or implants are inserted, in the bone at a pre-defined distance apart. The system and method may be particularly useful to assist in aligning a cut guide on a bone at a planned location. The system and method may be an improvement to existing robotic systems, navigation system, and/or procedural methods, in terms of accuracy, efficiency, and in some cases less hardware required to complete the procedure.

It is appreciated that while a femur and an associated cutting guide, in the context of a total knee arthroplasty (TKA) are used to illustrate the inventive system and method, other surgical procedures for joint replacements involving the knee, hip, ankle, shoulder, elbow, jaw, as well as for other structures in the body including the vertebra of the spine may benefit from the concepts and invention presented herein. Likewise, the embodiments described herein may be readily adapted for use in a myriad of applications where it is desirous to position implants for joint replacement procedures in other portions of the body.

The present invention will now be described with reference to the following embodiments. As is apparent by these descriptions, this invention can be embodied in different forms and should not be construed as limited to the embodiments set forth herein. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references mentioned herein are incorporated by reference in their entirety.

It is to be understood that in instances where a range of values are provided that the range is intended to encompass not only the end point values of the range but also intermediate values of the range as explicitly being included within the range and varying by the last significant figure of the range. By way of example, a recited range from I to 4 is intended to: 1-3, 1-2, 2-4, 3-4, and 1-4.

Definitions herein as set forth below.

As used in the description of the invention and the appended claims, the singular forms “a, an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, like reference numerals described with respect to subsequent drawings have the meaning imparted thereto with respect to the previously detailed drawings.

As used herein, the term “bone data” refers to data related to one or more bones. The bone data may be determined: (i) prior to making modifications (e.g., bone cuts, insertion of a pin or screw, etc.) to one or more bones, referred to as pre-operative bone data; and/or (ii) determined after one or more modifications have been made to a bone, referred to as postmodification bone data. The bone data may include: the shapes of the one or more bones; the sizes of the one or more bones; angles and axes associated with the one or more bones (e.g. inclination or anteversion of the glenoid); angles and axes associated with two or more bones relative to one another (e.g., the combined inclination and anteversion of the shoulder joint); anatomical landmarks associated with the one or more bones (e.g., humeral head center, greater tuberosity, lesser tuberosity, intertubercular groove glenoid center,); bone density data; bone microarchitecture data; and stress/loading conditions of the bone(s). By way of example, the bone data may include one or more of the following: an image data set of one or more bones (e.g., an image data set acquired via fluoroscopy, computed tomography (CT), magnetic resonance imaging (IVIRI), ultrasound, other x-ray modalities, laser scan, etc.); three-dimensional (3-D) bone models, which may include a virtual generic 3-D model of the bone, a physical 3-D model of the bone, a virtual patient-specific 3-D model of the bone generated from an image data set of the bone; and a set of data collected directly on the bone intra-operatively commonly used with imageless CAS devices (e.g., laser scanning the bone, painting the bone with a digitizer). The term “bone model” may refer to a full representation of a bone (e.g., a whole femur bone) or a partial representation of a bone (e.g., only a distal region or fragment of a femur bone). Likewise, the term “bone” may refer to a whole bone, a portion of the bone (e.g., distal portion of the femur), or a fragment of a bone. The term “virtual” may also be referred to herein as “digital”, meaning the data is stored, generated, and/or processed by a computer.

As used herein, the term “end-effector” refers to a tool, an instrument, or a distal end of a robot (e.g., a coupler for coupling a tool to the robot). The “end-effector” may be configured to couple to a robot, where the robot is directed to control movement of the end-effector in at least one degree-of-freedom. The “end-effector” may be configured to removably couple to the robot. Examples of “end-effectors” include: a pin (e.g., bone pin); a drill bit; a burr; an end-mill, a probe or pointer, a cutter; a saw-blade; a reamer; a broach; a laser; forceps or graspers; a screw (e.g., a pedicle screw, trauma screw); a nail; a coupler (e.g., chuck, collet, socket) for coupling a tool to the robot, or any other tool, instrument, coupler, or implant. The “end-effector” may be specifically designed to perform work on the environment, either directly (e.g., a drill bit making physical contact with the workpiece (e.g., bone)) or indirectly (e.g., the end-effector is a laser that emits a laser beam that contacts the workpiece).

As used herein, the terms “computer-assisted surgical device” and “CAS device’, refer to devices used in surgical procedures that are at least in part assisted by one or more computers. Examples of CAS devices illustratively include tracked/navigated instruments and surgical robots. Examples of a surgical robot illustratively include robotic hand-held devices, serial-chain robots, bone mounted robots, parallel robots, or master-slave robots, as described in

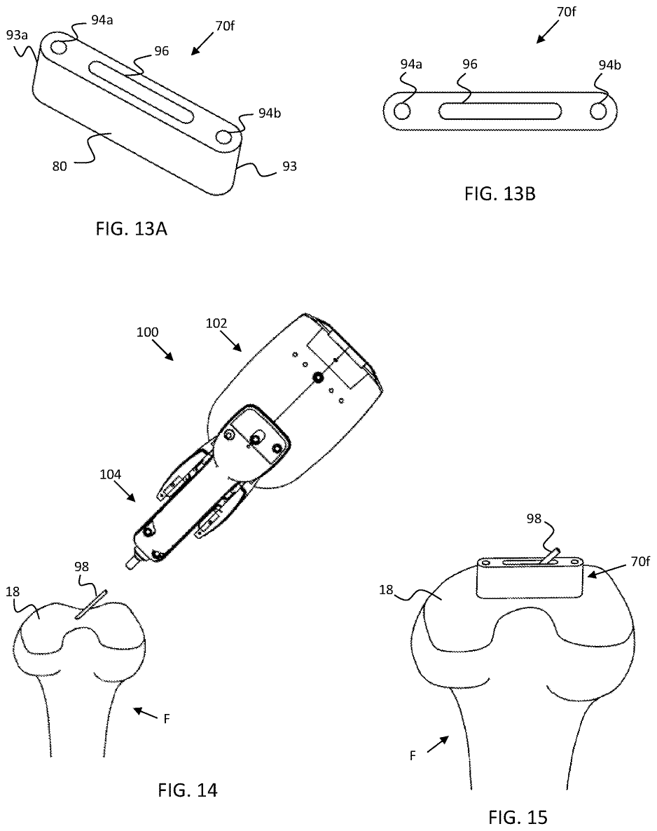

U.S. Pat. Nos. 5,086,401; 6,757,582; 7,206,626; 8,876,830; 8,961,536; 9,707,043; and 11,457,980; which patents and patent application are incorporated herein by reference. The surgical robot may be active (e.g., automatic/autonomous control), semi-active (e.g., a combination of automatic and manual control), haptic (e.g., tactile, force, and/or auditory feedback), and/or provide power control (e.g., turning a robot or a part thereof on and off). It should be appreciated that the terms “robot” and “robotic” are used interchangeably herein. The terms “computer-assisted surgical system” and “CAS system” refer to a system comprising at least one CAS device and may further include additional computers, software, devices, or instruments. An example of a CAS system may include: i) a CAS device and software (e.g., cutting instructions, pre-operative bone data) used by the CAS device; ii) a CAS device and software (e.g., surgical planning software) used with a CAS device; iii) one or more CAS devices (e.g., a surgical robot); iv) a combination of i), ii), and iii); and iv) any of the aforementioned with additional devices or software (e.g., a tracking system, tracked/navigated instruments, tracking arrays, bone pins, rongeur, an oscillating saw, a rotary drill, manual cutting guides, manual cutting blocks, manual cutting jigs, etc.). In particular embodiments of the present invention, the CAS device is a hand-held robotic device as described with reference to FIGS. 13, 14A, and 14B, which is particularly adapted for aligning an axis of a end-effector (e.g., pins, drill bit) coincident with a virtual plane for inserting at least a portion of the end-effector in the bone coincident with the virtual plane.

Also referenced herein is a “surgical plan.” A surgical plan is generated using planning software. The surgical plan may be generated pre-operatively, intra-operatively, or preoperatively and then modified intra-operatively. The planning software may be used to plan the location for an implant with respect to a bone and/or plan a location to make one or more modifications (e.g., bone cuts, location for inserting bone pins, location for forming holes) to the bone. The planning software may include various software tools and widgets for planning the surgical procedure. This may include, for example, planning: (i) a location for implant data (e.g., a 3-D implant model) with respect to bone data (e.g., a 3-D bone model) to define a location for the implant with respect to the bone; (ii) a location for one or more bone cuts (e.g., a location for sawing the bone, a location for forming holes) to be made relative to bone data to define the locations for one or more cuts to be made on the bone, and/or (iii) one or more locations for inserting hardware (e.g., bone pins, screws) relative to bone data. All of which may be used to define locations for device operating data (e.g., a cut-file, a virtual plane, virtual boundary, a virtual axis) with respect to the bone data, where a CAS device is directed to control movement of an end-effector (e.g., the hardware, a burr, end-mill, drill bit) with respect to the bone according to the device operating data. The device operating data may also be referred to as data used for operating a device. The device operating data may also, or alternatively, be used to provide feedback (e.g., display instructions or alignment data on a HUD or display monitor) to a user to assist the user in aligning an end-effector with a target axis as described herein

As used herein, the term “digitizer” refers to a device capable of measuring, collecting, recording, and/or designating the position of physical locations (e.g., points, lines, planes, boundaries, etc.) in three-dimensional space. By way of example but not limitation, a “digitizer” may be: a “mechanical digitizer” having passive links and joints, such as the highresolution electro-mechanical sensor arm described in U.S. Pat. No. 6,033,415 (which U.S. patent is hereby incorporated herein by reference); a non-mechanically tracked digitizer probe (e.g., optically tracked, electromagnetically tracked, acoustically tracked, and equivalents thereof) as described for example in U.S. Pat. No. 7,043,961 (which U.S. patent is hereby incorporated herein by reference); an end-effector of a robotic device; or a laser scanner.

As used herein, the term “digitizing” refers to the collecting, measuring, designating, and/or recording of physical locations in space using a digitizer. In some embodiments, “digitizing” may refer to the conversion of a designated location, area, or volume in space to a digital format. For example, a tracking system may determine the location of a digitizer probe tip in contact with a point on the bone, where the determined location of that point is saved to computer memory.

As used herein, the term “registration” refers to: the determination of the spatial relationship between two or more objects; the determining of a coordinate transformation between two or more coordinate systems associated with those objects; the mapping of an object onto another object; and a combination thereof. Examples of objects routinely registered in an operating room (OR) illustratively include: CAS systems/devices; anatomy (e.g., bone); bone data (e.g., 3-D virtual bone models); a surgical plan (e.g., location of virtual planes defined relative to bone data, cutting instructions defined relative to bone data, or other device operating data defined relative to bone data); and any external landmarks (e.g., a tracking array affixed to a bone, an anatomical landmark, a designated point/feature on a bone, etc.) associated with the bone (if such landmarks exist). Methods of registration known in the art are described in U.S. Pat. Nos. 6,033,415; 6,010,177; 8,036,441; 8,287,522; and 10,537,388. In particular embodiments associated with orthopedic procedures, the registration procedure relies on the manual collection of several points (i.e., point-to-point, point-to-surface) on the bone using a tracked digitizer where the surgeon is prompted to collect several points on the bone that are readily mapped to corresponding points or surfaces on a 3-D bone model. The points collected from the surface of a bone with the digitizer may be matched using iterative closest point (ICP) algorithms to generate a transformation matrix. This transformation matrix and various other transformation matrices provides the mathematical locational relationship between: (i) bone data (e.g., a 3-D bone model, planned location for forming one or more cut surfaces; planned location for an implant model relative to a bone model); and/or a surgical plan (e.g., a pre-defined location for a targeted virtual plane that was defined with respect to bone data, a pre-defined location for a targeted axis that was defined with respect to bone data, a pre-defined location of other device operating data that was defined with respect to bone data); and (ii) the coordinate system of a tracking array affixed to the bone (if present); and/or a CAS device (e.g., the base coordinate system of the CAS device, or a coordinate system of a tracking array affixed to the CAS device and, if needed, calibration data and/or kinematic data that define the location of an end-effector relative to the tracking array); and (iii) any other coordinate system or object required to perform the procedure. In other embodiments, the registration is performed using imageless registration.

As used herein, the term “display” is intended to encompass a variety of the digital devices that during operation provide an image (including multiple images in succession to form a video feed) recognizable to human viewing. Digital devices operative herein as displays illustratively include a graphical user interface (GUI), a computer or television (TV) monitor, a holographic display, a mobile display, a smartphone display, a video wall, a head-mounted display, a heads-up display (HUD), a virtual reality headset, a broadcast reference monitor, any of the aforementioned with touchscreen capabilities, and a combination thereof. One or more computers that may include an associated processor may be operatively coupled to the display for controlling the output of the display. In particular embodiments of the present invention, the “display” is a heads-up-display (HUD) provided on glasses that are worn on a user's head and positioned/oriented thereon for viewing by the user's eyes.

As used herein, the term “feedback” may refer to visual feedback provided on a display. This “feedback” may also be provided in lieu of or addition to visual feedback. For example, the “feedback” may include audio feedback, haptic/tactile feedback (e.g., a buzz or vibration when a digitizer tip is located at in an area of max deviation), or other visual feedback (e.g., a light on the digitizer may turn green or red depending on the amount of error between the digitizer tip and a planned cut surface).

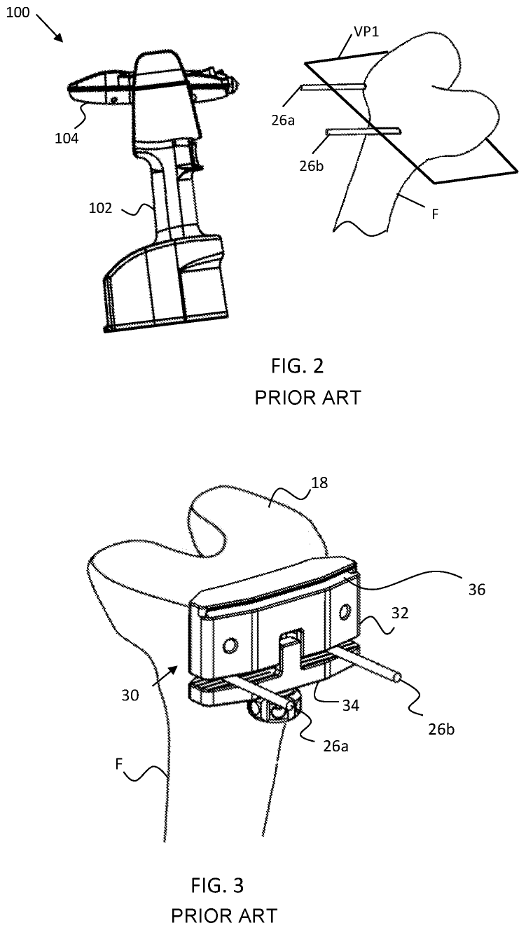

With reference now to the figures, FIGS. 2-6 depict a plurality of prior art steps for forming a plurality of cut surfaces on a bone during a TKA procedure with the assistance of a CAS system (e.g., a hand-held robotic system). The procedure may begin by planning the location for mounting the implants on the bone using planning software. The planning software may include models of the femur and tibia (also referred to as a femoral bone model or tibial bone model), which may have been generated from an image data set (e.g., CT scan data) of the patient's bones. In a particular embodiment, the planning software may include two planning modes. The first planning mode may include a library of 3-D implant models that are supported by the system to allow the user to plan the position and orientation (POSE) for mounting an implant relative to a bone such as in a conventional pre-operative planning manner. The second planning mode is for planning TKA procedures with an implant agnostic system (i.e., a system that lacks information, or is not programmed with, the full geometry data of the implant) by allowing the user (or system) to directly plan the location of the bone cuts for forming the cut surfaces on the bone as described in PCT publication WO2025049679. In the first planning mode, a graphical user interface (GUI) may include a drop-down menu for a user to select a desired implant make, model, and size. The GUI may then display the selected implants in the form of a femoral implant model relative to a femoral bone model and a tibial implant model relative to a tibial bone model. The GUI may include a plurality of software tools, or widgets, that allow the user to adjust the position and orientation of the implant models relative to the bone models to designate the best fit and alignment for mounting the implant onto the bone. In other embodiments, the planning software may automatically determine a position and orientation for mounting the implant on bone based on a user's planning preferences/philosophy (e.g., neutral mechanical axis alignment vs. kinematic alignment). The planned location for mounting the implant model relative to the bone model defines the planned locations for forming the cut surfaces on the bone (e.g., the planned location for forming distal cut surface is known based on where the distal contact surface of the femoral implant model overlaps with the femoral bone model). The GUI may display graphics (e.g., line, box, grayed out bone areas) showing the planned locations of the cut surfaces, or the bone models may be updated show the cut surfaces directly on the bone models. The GUI may also display alignment information in relevant clinical degrees-of-freedom to assist in planning the position for mounting the implant on the bone. This information may include the degrees of varus-valgus of the bone with respect to the mechanical axis as a result of a planned distal cut surface, the degrees of external-internal rotation of the implant from a bone condylar axis (e.g., transepicondylar axis (TEA), posterior condylar axis (PCA)) as a result of the planned posterior cut surface, the degrees of flexionextension of the implant with respect to the bone as a result of the planned distal cut surface and/or planned posterior cut surface, the tibial posterior slope of the implant with respect to the bone as a result of the planned tibial cut, etc. The GUI may also display an amount of bone that will be resected for a given bone cut (e.g., 7.0 mm on the lateral distal condyle and 4.5 mm on the medial distal condyle). After the user is satisfied with the planned location for forming the bone cuts, the plan is saved for use by the CAS system.

The planning software may also define the locations of virtual planes relative to the bone models. As shown in prior art FIG. 2, the location of a first virtual plane ‘VI) l’ may be defined relative to the planned location for forming the distal cut surface 18. The planning software is programmed with the geometry data about the cut guide 30 (shown in FIG. 3), particularly the distance between the location of the cut guide guide slot 36 and the location where the cut guide 30 will be coupled (e.g., clamped, press-fit) onto the pins (26a, 26b) inserted in the bone. The system may translate a plane corresponding to the planned location for forming the distal cut surface by this known distance to define the location of the first virtual plane ‘VP’ relative to the bone model, such that when the cut guide 30 is coupled onto the pins (26a, 26b) inserted in the bone, the guide slot 36 aligns with the planned location for forming the distal cut surface.

In the operating room (OR), the femoral bone model is registered to the femur in a coordinate system of a tracking array affixed to the femur, which allows the system to determine the locations of any planning data (e.g., the location of the first virtual plane ‘VPI’ defined relative to the femoral bone model) relative to the real-time location of the femur. As shown in prior art FIG. 2, the hand-held robotic device 100 (referred to hereinafter as hand-held robot) is used to insert two pins (26a and 26b) into the bone at locations coincident with the first virtual plane ‘VPI’. Details of the hand-held robotic system and the method for using the hand-held robot 100 for TKA procedures is described in U.S. Pat. No. 11,457,980. Briefly, the hand-held robot 100 includes a working portion 104 movably coupled to a hand-held portion 102 and an actuator system for moving the working portion 104 relative to the hand-held portion 102. The working portion drives or operates an end-effector (e.g., rotating a pin, rotating a drill bit, oscillating a saw blade, etc.) while the actuator system moves the working portion 104 relative to the hand-held portion 102 in response to control signals to maintain alignment of an axis of the end-effector coincident with a virtual plane. The locations of the hand-held robot 100 (and more specifically the location of the end-effector or end-effector axis) and the location of the virtual plane ‘VI)’ are determined in real-time by a computing system using tracking data from a tracking system. Therefore, the system can adjust the position of the end-effector to maintain alignment of the end-effector axis coincident with the virtual plane ‘VPI’ to compensate for: (i) the user's hand movements while wielding the handheld robot 100; (ii) any movement of the bone; and/or (iii) any other movements that may cause the end-effector axis to deviate out of coincidence with a virtual plane. While the system maintains alignment of the end-effector-axis coincident with a virtual plane, the user manually advances the hand-held robot 100 towards the bone to insert at least a portion of the end-effector (e.g., a pin) in the bone coincident with the virtual plane. Therefore, the final location of where the end-effector is inserted in the bone coincident with the virtual plane is at the discretion of the user. For example, the pins (26a and 26b) shown in prior art FIG. 2 may be inserted in the bone at any medial-lateral location that is coincident with the first virtual plane ‘VPI.

After the hand-held robot 100 is used to insert the two pins (26a, 26b) in the bone coincident with the first virtual plane ‘VPI a cut guide 30 is coupled to the pins (26a, 26b) as shown in prior art FIG. 3. The cut guide 30 may include a top portion 32, a bottom portion 34, and a fastening mechanism (e.g., clamping mechanism) that moves the bottom portion 34 relative to the top portion 32 to secure the cut guide 30 to the pins (26a, 26b). The cut guide 30 further includes a guide slot 36 for receiving a surgical saw blade to guide the surgical saw blade in forming the distal femoral cut surface 18. As previously mentioned, the pins (26a, 26b) are placed in the bone at a location that aligns the guide slot 30 of the cut guide 30 with the planned location for forming the distal cut surface when the cut guide 30 is coupled to the pins (26a, 26b). After the distal femoral cut surface 18 is formed with a surgical saw blade, the cut guide 30 and pins (26a, 26b) may be removed from the bone.

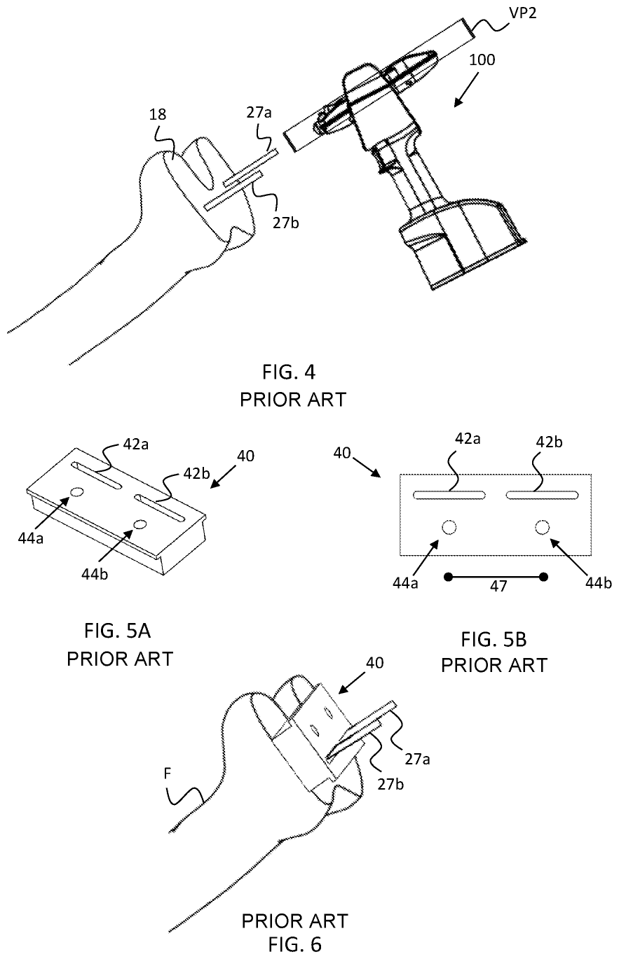

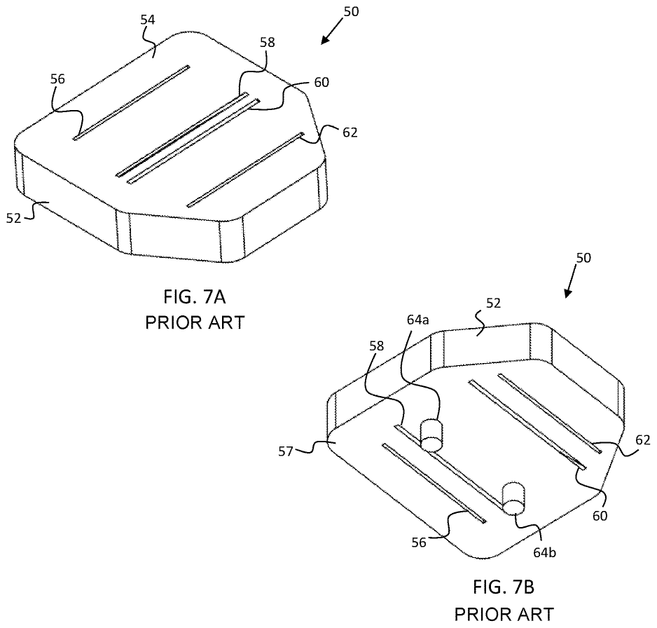

With reference to prior art FIGS. 4-6, the next step in the procedure is forming the remaining cut surfaces on the distal femur. The hand-held robot 100 is first used to maintain alignment of an axis of a pin 27a coincident with a second virtual plane ‘VP2’ for inserting pins (27a, 27b) in the distal cut surface 18 coincident with the second virtual plane ‘VP2.’ A drill guide 40 (e.g., as shown in prior art FIGS. 5A and 5B) is then coupled to the pins (27a, 27b), as depicted schematically in prior art FIG. 6. The drill guide 40 includes a pair of pin slots (42a, 42b), where the pin slots (42a, 42b) slide onto the pins (27a, 27b) inserted in the distal cut surface 18. The drill guide 40 further includes two guide holes (44a, 44b) for receiving a drill bit to guide the formation of two peg holes. The distance between the guide holes (44a, 44b) (as represented by line 47 in prior art FIG. 5B) is equal to the distance between the pegs (64a, 64b) of the 4-in-l block 50 (as represented by line 65 in FIG. 7B). After the drill guide 40 is coupled to the pins (27a, 27b) inserted in the distal cut surface 18, the user may form the peg holes in the distal cut surface 18 by advancing a drill bit through the guide holes (44a, 44b). The location of the second virtual plane ‘VI)2’ and the subsequent insertion of the pins coincident with that second virtual plane ‘VI)’ is defined such that the guide holes (44a, 44b), and subsequent formation of the peg holes through the guide holes (44a, 44b), aligns the guide slots of the 4-in-l cut block 50 (see prior art FIGS. 7A and 7B) on the bone with the planned location for forming the remaining cut surfaces on the bone. To define the location of the second virtual plane ‘VP2,’ the system may first identify the planned location for forming the posterior cut surface 22 on the femur “F” based on the surgical plan. Then, the system determines the location for forming the peg holes using the geometry of the 4-in-l cut block 50. For example, the system may anteriorly translate a first plane that corresponds to the planned location of the posterior cut surface by the known (or programmed) anterior-posterior distance between the posterior guide slot 56 of the 4-in-l cut block 50 and the center of the pegs (64a, 64b). The system may then anteriorly (or posteriorly) translate that first plane again (from its new location, which is the location for the peg holes) by the known (or programmed) anteriorposterior distance between the center of the guide holes (44a, 44b) and the center of the slots (42a, 42b) to ultimately define the location for the second virtual plane ‘VP2.’ Thus, when the pegs (64a, 64b) of the 4-in-l block 50 is placed in the peg holes, the posterior guide slot 56 is aligned with the planned location for forming the posterior cut surface 22. All the other guide slots (e.g., anterior guide slot) are also aligned in the planned POSE because the geometry of the 4-in-l block is based on the geometry of the implant.

With reference now to prior art FIGS. 7A and 7B, after the peg holes are formed in the distal cut surface 18, the pegs (64a, 64b) of the 4-in-l cut block 50 are placed in the peg holes formed on the distal cut surface, and the remaining cut surfaces are formed by advancing a surgical saw blade through each of the guide slots. The 4-in-l cut block 50 may include body 52 having a top surface 54, a bottom surface 57, a pair of pegs (64a, 64b) extending from the bottom surface 57, and a plurality of guide slots. The 4-in-l cut block 80 in some embodiments includes an anterior guide slot 62, an anterior chamfer guide slot 60, a posterior chamfer guide slot 58, and a posterior guide slot 56, although other configurations may be provided in alternative embodiments. Each of the guide slots receive a surgical saw blade for guiding the surgical saw blade in forming the corresponding cut surfaces on the bone. After all the cut surfaces are formed on the distal femur, the femoral implant may be mounted onto the cut surfaces to complete the femoral side of the TKA procedure.

The above procedure (described with reference to prior art FIG. 2-FIG. 7B) for forming all five cut surfaces on the distal femur with the hand-held robotic system is particularly efficient and accurate. Nonetheless, there is a recognition that the steps between placement of the pins (27a, 27b) in the distal cut surface and the placement of the N-in-l cutting block may be improved. In addition, other robotic systems, navigation systems, and procedural methods may likewise benefit from the devices and methods described hereinafter for different applications and purposes. For example, the device and methods described hereinafter may be used for positioning an end-effector (e.g., pins, drill bit, burr, pedicle screws), and the like at a predefined location, where two or more end-effectors need to be inserted into a bone at locations that are a pre-defined distance apart. This may be applicable in spine surgery when placing pedicle screws on both sides of a vertebra, where the distance between the pedicles are known (e.g., the distance is pre-defined based on bone data). The following devices and methods may be used to insert pedicle screws in the pedicles located on both sides of the vertebra by that known distance. Furthermore, the devices and method described hereinafter may be particularly advantageous for use with a robotic device that operates (or is controllable) in less than number of degrees-of-freedom required to position two or more tools or implants a pre-defined distance apart. For example, a hand-held robot that operates in two degrees-of-freedom (e.g., one translational degree-of-freedom and one rotational degree-of-freedom) may lack a controllable degree-of-freedom for spacing two or more end-effectors (e.g., pins, drill bit, burr, pedicle screw) a pre-defined distance apart. The following devices and methods may be used with said hand-held robot to accurately space the end-effectors.

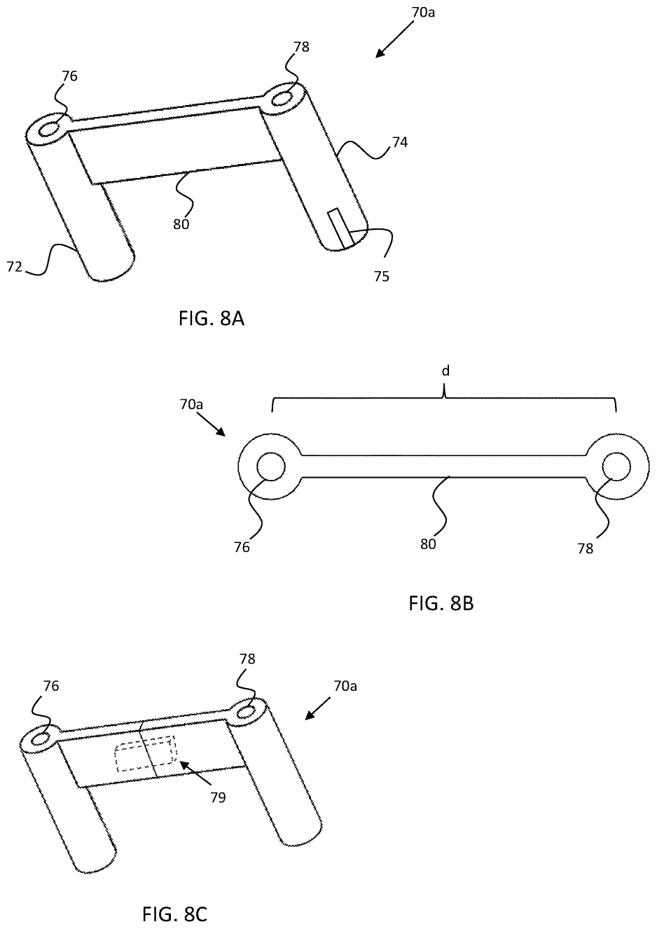

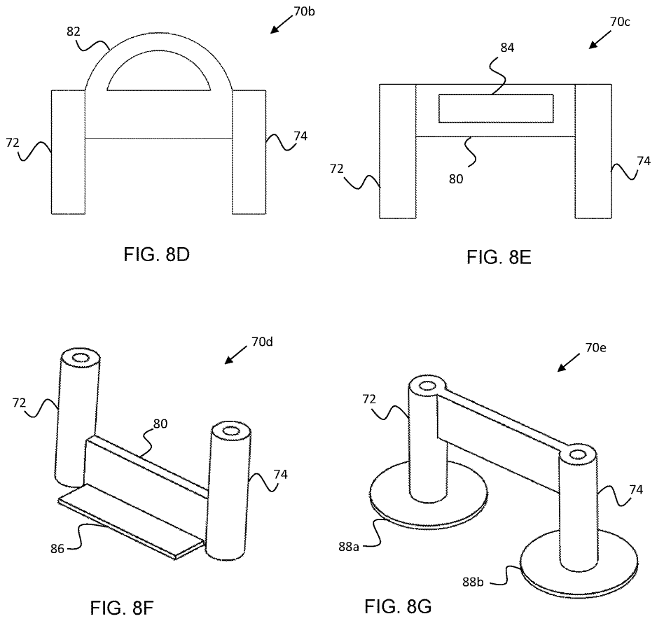

As shown with respect to FIGS. 8A-l l, a system and method for forming features (e.g., peg holes), or inserting implants, at locations in the bone and at a pre-defined distance apart is shown. The example shown in FIGS. 8A-l I is with respect to the formation of peg holes in the bone at locations that are a pre-defined distance apart, where that pre-defined distance corresponds to the distance between the pegs of a 4-in-l cut block. This allows the 4-in-l cut block to be mounted on the bone in a planned location for guiding the formation of cut surfaces on the bone. The system includes an inventive drill guide 70a as shown in FIGS. 8A and 8B, which can replace the aforementioned drill guide 40 and the steps associated therewith (as shown in prior art FIGS. 4-6) to streamline the process of forming the peg holes in the distal cut surface 18. The drill guide 70a includes a pair of guide tubes (72, 74) for guiding the formation of each peg hole in the bone needed to mounted a pre-selected N-in-l cut block to the bone surface. While two guide tubes (72, 74) are shown, it is appreciated that more guide tubes may be provided and the guide tubes (72, 74) need not be in a linear arrangement. The guide tubes (72, 74) are interconnected by a cross-bar portion 80 (or elongated member). The guide tubes (72, 74) each define a bore (76, 78) having a pre-selected diameter or pre-selected width. In some inventive embodiments, the bores (76, 78) of the guide tubes (72 and 74) are parallel. The diameter (or width) of each bore (76, 78) is dimensioned to accommodate an end-effector (e.g., a pin, drill bit, pedicle screw, or other surgical boring tool) such that at least a portion of the end-effector can be inserted through each bore (76, 78). In other words, the diameter (or width) of each bore (76, 78) is dimensioned to accommodate an end-effector such that the bore (76 and 78) can receive at least a portion of an end-effector therethrough. The diameter (or width) of the bore (76 and 78) may further be dimensioned to create a preselected margin between the end-effector and the inner wall of the bore (76, 78) when the end-effector is inserted therein. The preselected margin between the inner wall and end-effector is selected based on factors that include limiting the endeffector from walking or wobbling within the bore, and accommodating bone debris generated by the boring operation. In some inventive embodiments, a vent 75 is formed in each guide tube (72, 74). The vent 75 may be positioned proximal to a bone contacting surface (e.g., distal cut surface 18 when the drill guide 70a is in contact with the distal cut surface 18) or along the length of the guide tube (72, 74). The vent provides a controlled area of bone debris and thereby precludes bone debris from clogging the bore (76, 78) and/or preselected margin.

The drill guide 70a is configured to guide the formation of features, or guide the insertion of implants, in a bone at a pre-defined distance apart, an example of which is further described below. This is accomplished based on the spacing between the center of the bores (76, 78). FIG. 8B depicts said distance ‘d’. The distance ‘d’ between the bores (76, 78) is dependent on the length of the cross-bar 80 connecting the first guide tube 72 and the second guide tube 74 The length of the cross-bar 80 may be defined prior to manufacturing the drill guide 70, or in some embodiments, the length of the cross-bar 80 may be adjustable by an adjustment mechanism 79 (e.g., a telescoping mechanism, worm gears, rack and pinion, interlocking clips, ball-detent or other spring-based mechanism, etc.) as shown in FIG. 8C. The distance between the bores (76, 78) may be pre-defined prior to the surgical procedure depending on the distance required for forming the features, or inserting the implants, in the bone. For example, the spacing between the pegs of a 4-in-l cut block may be known prior to the TKA procedure. A drill guide 70a may be manufactured having bores (76, 78) spaced a distance ‘d’ apart corresponding to the known distance between the pegs (64a, 64b) of the 4-in-l cut block 50. If the drill guide includes an adjustment mechanism 79, the user may adjust the distance between the bores (76, 78) to correspond to the distance between the pegs of a selected cut block in the operating room. In some embodiments, a kit (or tray) of drill guides is provided during the surgical procedure, where each drill guide 70a in the kit has different spacing between the bores (76, 78). In the operating room (OR), the user then selects a drill guide 70a in the kit that has a bore spacing corresponding to the spacing between the pegs (64a, 64b) of a chosen 4-in-l cut block 50.

The drill guide 70a further ensures that the features are formed in the bone normal to a bone surface (e.g., distal cut surface 18). Likewise, the drill guide 70a ensures that any implants inserted in the bone are inserted normal to a bone surface. This is particularly important when a the feature or implant needs to be oriented normal to a bone surface (e.g., the peg holes need to be formed in the bone normal to the distal cut surface in order to receive the pegs of a 4-in-l cut block in the proper orientation). In particular embodiments, the surfaces of the guide tubes (72, 74) that contact the bone surfaces are planar, or flat, to make contact with a planar cut surface (e.g., distal cut surface 18). As such, the guide tubes (72, 74) when placed in contact with a planar cut surface will orient the guide tubes (72, 74) normal to the planar cut surface.

The drill guide 70a is formed of a suitable material for contact with subject bone tissue and to constrain a surgical tool operating therein from deviating from a path defined by the bore. Such materials illustratively include stainless steel, titanium, tantalum, tungsten carbide, and other alloys, which may be inclusive of any of the aforementioned elements. The drill guide 70a may further be formed of polymeric materials known in the art. The drill guide 70a may be sterilizable such that the drill guide 70a may be used in multiple procedures, while in other embodiments, the drill guide 70a may be a single-use disposable device.

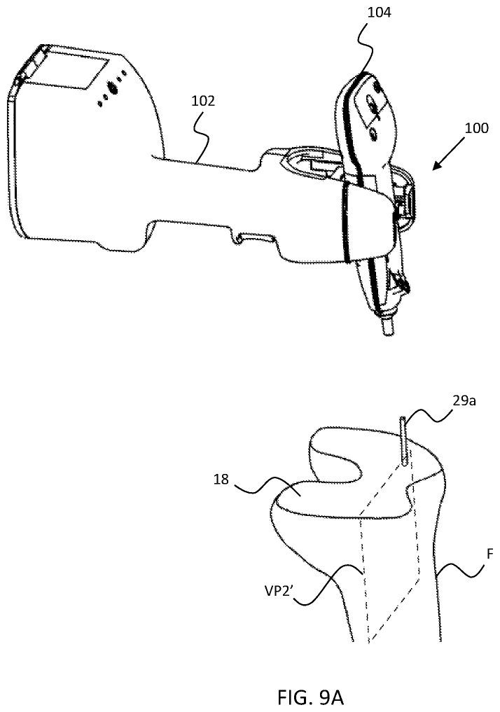

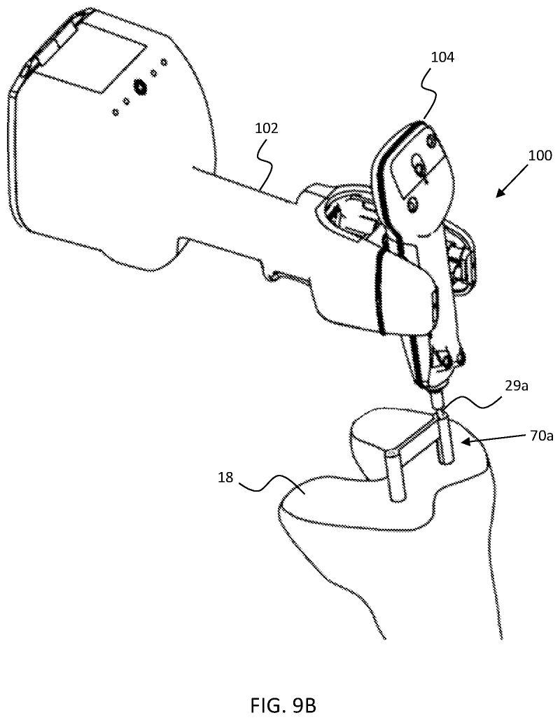

As shown in FIGS. 8C-8D in which like reference numerals have the meanings ascribed thereto with respect to FIGS. 8A and 8B, various embodiments of a drill guide having stabilizing features are provided. The stabilizing features function to limit unwanted to movements of the drill guide relative to bone surface (e.g., distal cut surface 18) when forming features, or inserting implants, in the bone. In some instances, the stabilizing features render the drill guide hands free during formation of a second feature, or insertions of a second implant, in the bone as further described below. FIG. 8C depicts an embodiment where the drill guide 70b includes a handle 82 configured to be held by a user (e.g., surgeon, a surgical technician), or configured to be coupled to another tool (e.g., a clamp) to stabilize the drill guide 70b during use. FIG. 8D depicts an embodiment where the cross-bar 80 include an aperture 84 configured to be held by a user (e.g., surgeon, a surgical technician), or configured to be coupled to another tool (e.g., a clamp) to stabilize the drill guide 70c during use. FIG. 8E depicts an embodiment where the drill guide 70d includes one or more bone contacting flange(s) 86 to stabilize the drill guide 86 on a bone surface during use. The flange(s) 86 may extend laterally from the cross-bar 80 and prevent the drill guide 70d from rotating relative to the bone surface during use. The flange(s) 80 may further assist in maintaining the guide tubes (72, 74) normal to the bone surface. FIG. 8F depicts an embodiment where the drill guide 70e includes bone contacting flanges (88a, 88b) positioned on the end of each guide tube (72, 74). The bone contacting flanges (88a, 88b) likewise prevent the drill guide 70e from rotating relative to the bone surface during use and may further assist in maintaining the guide tubes (72,74) normal to the bone surface. In other embodiments, the drill guide may further include a clamp or other coupling mechanism that couples directly to the bone to stabilize movement of the drill guide relative to the bone surface. [00611 An exemplary procedure for the use of a drill guide 70 is shown in FIGS. 9A-l l. The procedure may begin as previously described with respect to prior art FIGS. 2 and 3. For example, a surgical plan is created to define the planned location for mounting an implant on the bone. Planning software defines the location for a first virtual plane ‘VI) I’ for inserting pins (26a, 26b) in the bone coincident with the first virtual plane ‘VI) I’. A hand-held robot 100 assists the user in inserting the pins (26a, 26b) in the bone coincident with the first virtual plane ‘VI) I A cut guide 30 is then coupled to the pins (26a, 26b) and a user manually advances a saw blade through the guide slot 36 of the cut guide 30 to form the distal cut surface 18 on the bone. Then, with respect to FIG. 9A, the hand-held robot 100 is used to insert a first pin 29 in the bone coincident with a second virtual plane ‘VP2”. The second virtual plane ‘VP2” is defined relative to the bone by the planning software using the geometry of the 4-in-l cut block 50. The second virtual plane ‘VP2” is defined coincident with the location for forming the peg holes in the distal cut surface 18. More particularly, the second virtual plane ‘VP2” may be defined, for example, by the planning software by: (i) determining the planned location for forming the posterior cut surface on the bone based on the planned location of the implant model positioned relative to the bone model (e.g., where the posterior contact surface of the implant model intersects the bone model when the implant model is positioned at its planned (or desired) location); (ii) determining the slot-to-peg distance, which is the distance between the posterior guide slot 56 of the 4-in-l cut block 50 and the center of the pegs (64a, 64b) (this may be a stored value in the planning software based on the geometry of the 4-in-l cut block 50); and (iii) anteriorly translating a virtual plane from the planned location for forming the posterior cut surface by the slot-to-peg distance to define the location for the second virtual plane ‘VP2”. The orientation of the second virtual plane ‘VP2” may therefore be parallel (and in some embodiments, co-planar) with a plane intersecting the center of both pegs of the 4-in-l cut block 50. It will be appreciated that the defined location of this second virtual plane ‘VP2” (as shown in FIG. 9) is different than the location of the second virtual plane ‘VP2’ as described with reference to prior art FIG. 4, in that the second virtual plane ‘VP2” (as shown in FIG. 9A) is coincident with the planned location for forming the peg holes.

The hand-held robot 100 maintains alignment of the first pin axis coincident with the second virtual plane ‘VP2” While the hand-held robot 100 maintains the alignment, the user manually advances the hand-held robot 100 towards the bone to insert the first pin 29a in the bone (and more particularly the distal cut surface 18). It should be appreciated that the user may select any medial-lateral location (relative to the bone) to insert the first pin 29a in the distal cut surface 18 that is coincident with the second virtual plane ‘VP2”. The diameter of the first pin 29a may be dimensioned to have the same or a slightly larger diameter than the diameter of a peg (64a, 64b) of the 4-in-l cut block 50 such that the first pin 29a (when removed from the bone) forms a peg hole in the distal cut surface 18 capable of receiving a peg (64a, 64b) of the 4-in-l cut block 50 therein. In some inventive embodiments, the first pin 29a is inserted through the distal cut surface 18 of the bone B to a depth sufficient to serve as a first peg hole complementary to a first peg of the N-in-l cutting block. The depth for inserting the first pin 29a in the bone may be controlled based on the height of the guide tubes (72, 74). For example, the guide tubes (72, 74) may be designed with a specific pre-defined height such that when the user is inserting the first pin 29a into the bone, the top surface of the guide tube (72, 74) will act as a stop to prevent the user from advancing the first pin 29a any deeper into the bone when the distal end (e.g., distal end of the chuck) of the hand-held robot 100 makes contact with the top surface of the guide tube (72, 74). In other inventive embodiments, the peg hole formed by the first pin 29a is enlarged and/or deepened to receive a peg (64a, 64b) of the 4-in-l cut block 50. After the first pin 29a is inserted in the bone, the first pin 29a is decoupled from the hand-held robot 100.

FIG. 9B depicts an alternative embodiment for inserting a first pin 29a into the distal cut surface 18 of the bone. In this embodiment, the drill guide 70a is used while inserting the first pin 29a into the distal cut surface 18 to ensure that the first pin 29a is inserted into the bone normal to the distal cut surface 18. The formation of the peg holes normal to the distal cut surface 18 may be particularly important to ensure that the 4-in-l cut block lies flush with distal cut surface 18 when the pegs (64a, 64b) are placed in the formed peg holes. As in FIG. 9A, the hand-held robot 100 maintains alignment of the first pin axis coincident with the second virtual plane ‘VP2”, except now the first pin 29a is also positioned in a guide tube (72, 74) of the drill guide 70a. While the hand-held robot 100 maintains this alignment and the first pin 29a is positioned in the guide tube (72, 74), the user manually advances the hand-held robot 100 towards the bone to insert the first pin 29a in the distal cut surface 18. During the process, the drill guide 70a may be stabilized relative to the bone by at least one of: (i) one of the stabilizing features described in FIGS. 8C-8F; (ii) the user, or an assistant user (e.g., surgical technician), holding the drill guide 70a; and/or (iii) resting or pinning the drill guide 70a against the distal cut surface 18

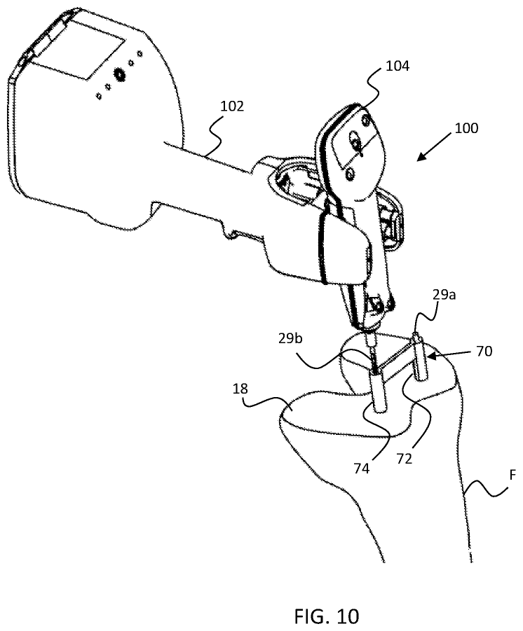

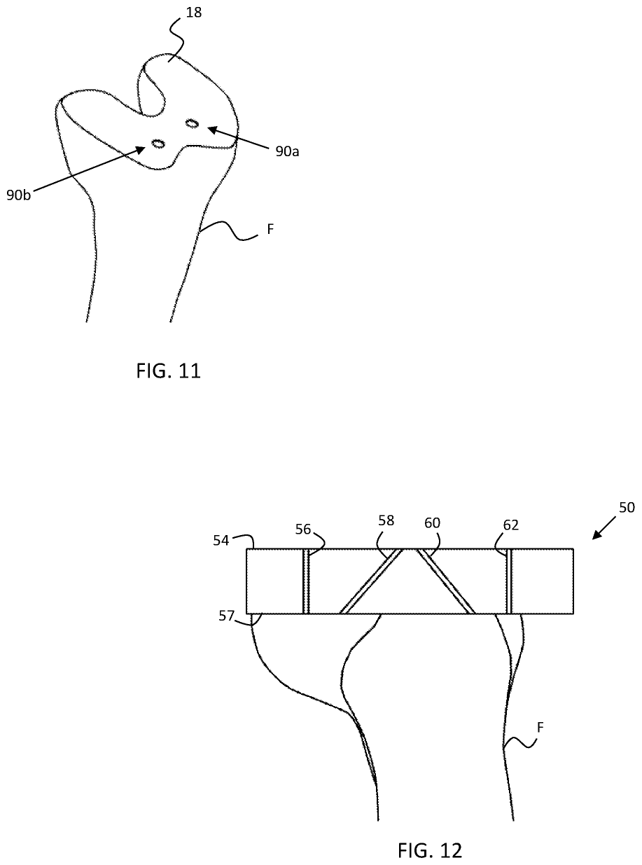

After the first pin 29a is inserted in the bone, a second pin 29b is inserted laterally offset therefrom as shown in FIG. 10. First, the drill guide 70a is coupled to the first pin 29 (if not done so already) by sliding the first bore 76 over the first pin 29a inserted in the bone. The first pin 29a inserted in the bone may act as an anchor point for the first guide tube 72, which prevents the drill guide 70 from translating relative to the bone yet permitting the drill guide 70 to pivot about the first pin 29a while the second pin 29b is inserted in the bone. A second pin 29b is then coupled to the hand-held robot 100. A portion of the second pin 29b is inserted into the second bore 78 of the second guide tube 74. The hand-held robot 100 then maintains alignment of the second pin axis coincident with the second virtual plane ‘VP2” (while the second pin 29b is positioned inside the second bore 78). While the hand-held robot 100 maintains this alignment, the user manually advances the second pin 29b towards the distal cut surface 18 to insert the second pin 29b in the distal cut surface 18. While the first pin 29a may be inserted at any mediallateral location (relative to the bone) in the bone that is coincident with the second virtual plane ‘VP2”, the drill guide 70 advantageously ensures that the second pin 29b is inserted in the bone at a pre-defined distance from the first pin 29a (i.e., the distance ‘d’ between the bores (76, 78) of the drill guide 70). In other words, the drill guide 70 restricts the user from manually moving the hand-held robot 100 in the medial-lateral direction (relative to the bone) to ensure the second pin 29b is inserted in the bone at a pre-defined distance (i.e., the distance between the bores (76, 78)) from the first pin 29a. Thus, having the spacing between the bores (76, 78) equal to the spacing of the pegs (64a, 64b) of the 4-in-l cut block 50 ensures that the spacing between the first pin 29a and the second pin 29b inserted in the bone will also equal the spacing between the pegs (54a, 64b) of the 4-in-l cut block 50.

The second pin 29b may have the same diameters with respect to the first pin 29a such that the second pin 29b (when removed from the bone) forms a peg hole in the bone capable of receiving a peg (64a, 64b) of the 4-in-l cut block 50.

After the first pin 29a and second pin 29b are inserted in the bone, the drill guide 70 and the pins (29a, 29b) may be removed. Removal of the first pin 29a and the second pin 29b from the bone reveal peg holes (90a, 90b) formed in the distal cut surface 18 suitably placed to receive the pegs (64a, 64b) of the 4-in-l cut block 50 as shown in FIG. l l. FIG. 12 depicts a cross-sectional view of the 4-in-l cut block 50 mounted to the distal cut surface 18, and positioned thereon in a planned location by inserting the pegs (64a, 64b) of the 4-in-l cut block 50 in the formed peg holes (90a, 90b). The user may then advance a saw blade through the guide slots to form the remaining cut surfaces on the bone in the planned locations. The implant 12 is then mounted on the bone to complete the procedure.

In a particular embodiment, the second pin 29b is replaced with a drill bit (or other bone removing tool) to directly form the second peg hole 90b in the bone. For example, the first pin 29a may be inserted into the distal cut surface 18 and the drill guide 70 assembled to the first pin 29a as described above with reference to FIGS. 9A and 9B. Then, a drill bit is coupled to the hand-held robot 100 instead of a second pin 29b. A portion of the drill bit is inserted into the second bore 78 of the second guide tube 74. The hand-held robot 100 then maintains alignment of the drill bit axis coincident with the second virtual plane ‘VP2” (while the drill bit is simultaneously positioned inside the second bore 78). While the hand-held robot 100 maintains this alignment, the user manually advances the drill bit towards the distal cut surface 18 to directly form the second peg hole 90b in the distal cut surface 18. This may save additional time in the OR because there is now no need to remove the second pin 90b from the bone to reveal the second peg hole 90b.

The use of a drill guide 70 according to the present invention, eliminates a series of steps that approximately correspond to the steps described with reference to prior art FIGS. 4-6. In particular, the drill guide 40 shown in prior art FIGS. 5A-5C is coupled to pins inserted in the bone and then a separate drill is used to form the peg holes by advancing a drill bit through the guide holes (44a, 44b) of the drill guide 40. In embodiments of the present invention, the peg holes are formed directly by the placement of the pins (29a, 29b) in the bone where the drill guide 70 ensures the pins (29a, 29b) are spaced the correct distance apart to accommodate the pegs (64a, 64b) of the 4-in-l cut block 50. There is no need for a separate drilling process.

### Aligning a Drill Guide Relative to a Bone With a Single Pin

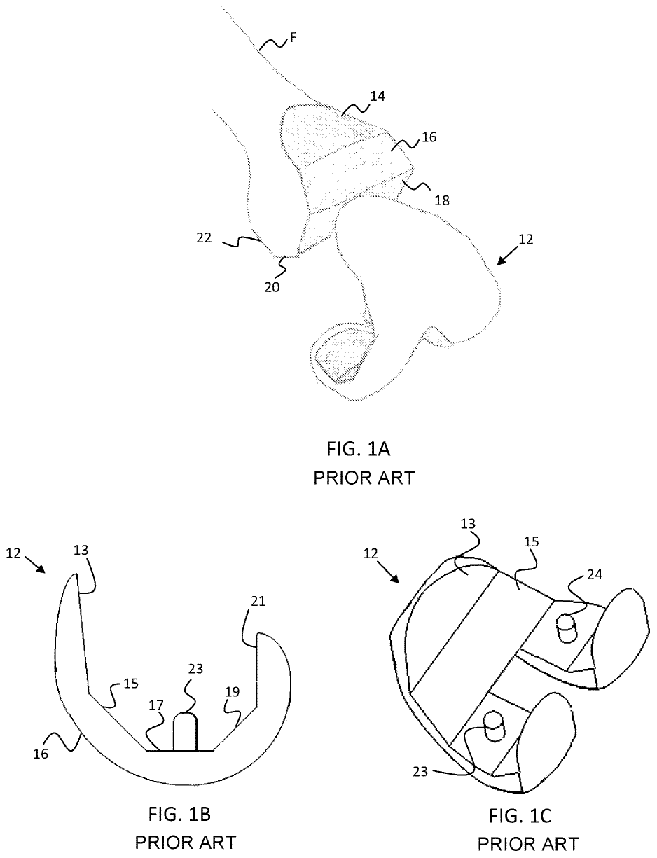

With reference to FIGS. 13A-15, a particular embodiment of a system and method for forming features (e.g., peg holes), or inserting implants, at locations in a bone and at a predefined distance apart is shown where only one pin is needed to align a drill guide 70f relative to the bone. FIGS. 13A and 1B depict a drill guide 70f having a first guide tube 93a with a first bore 94a, a second guide tube 93b with a bore 94b, a cross-bar 80 (or elongated member) interconnecting the first guide tube 9a and the second guide tube 93b, and a slot 96 (or opening) within the cross-bar 80 and positioned between the first guide tube 93a and the second guide tube 93b. The first bore 94a and second bore 94b are spaced a pre-defined distance apart for forming features, or inserting implants (e.g., pins), in the bone. For example, the distance between the first bore 94a and the second bore 94b may correspond to the distance between the pegs (64a, 64b) of a 4-in-l cut block 50. The drill guide 70f may include an adjustment mechanism for a user to adjust the distance between the first bore 94a and second bore 94b, or the drill guide 70f may be manufactured with the desired spacing, the same as described above with reference to the drill guides shown in FIGS. 8A-8F. Likewise, the drill guide 70f may include any of the stabilizing features described with reference to FIGS. 8C-8F.

A procedure for forming one or more features, or inserting implants, in the bone with drill guide 70f is shown in FIGS. 14 and 15. First, a second virtual plane ‘VP2” is defined relative to the bone, which is defined at the same location (and in the same manner) as the second virtual plane ‘VP2” described with reference to FIG. 9A. Next, a pin 98 is coupled to the hand-held robot 100. The hand-held robot 100 then maintains alignment of the pin axis coincident with the second virtual plane ‘VP2” While the hand-held robot 100 maintains this alignment, the user manually advances the pin 98, at an angle relative to the distal cut surface 18, to insert the pin 98 in the distal cut surface 18 at said angle as best seen in FIG. 14. To accomplish this, the user may be instructed by the CAS system to manually angle the hand-held robot 100 such that the pin 98 will be inserted into the distal cut surface 18 at said angle relative to the distal cut surface 18. For example, the CAS system may instruct the user to insert the pin 98 at an angle ranging from 10° to 80° relative to the distal cut surface 18, where 90° is perpendicular (or normal) to the distal cut surface 18. The user would then manually angle the hand-held robot 100 to insert the pin 98 into the distal cut surface 18 at an angle within that range. In particular embodiments, the range of the angle is between 10° and 55°. The hand-held robot 100 maintains the alignment of the pin axis coincident with the second virtual plane ‘VP2” to ensure the pin 98 is inserted in the distal cut surface 18 coincident with the second virtual plane ‘VP2” even with the pin 98 angled relative to the distal cut surface 18.

After the pin 98 is inserted in the bone, the drill guide 70f is positioned on the distal cut surface 18 by sliding the drill guide slot 96 over the pin 98. The angled pin inserted in the bone prevents the drill guide 70f from rotating and translating (except potential translations in the medial and lateral direction which is acceptable), locking in all the necessary degrees-of-freedom to align the drill guide 70f relative to the bone for forming the peg holes (94a, 94b) (a shown in FIG. I l) in the bone to receive the pegs (64a, 64b) of the 4-in-l cut block 50. In other words, the angled pin provides at least two spaced contact points with the inner walls of the drill guide slot 96 to prevent the drill guide 70f from rotating internally or externally relative to the distal cut surface 18. Therefore, after the drill guide 70f is coupled to the pin 98 (via the slot 96), the user may use a standard drill, or the hand-held robot 100 with a drill bit coupled thereto, to form the peg holes (90a, 90b) by advancing the drill bit through the first bore 94a and then the second bore 94b. The drill guide 70f and pin 98 may then be removed from the bone, and the pegs (64a, 64b) of the 4-in-l cut block 50 positioned in the formed peg holes (90a, 90b) to mount the 4-in-l cut block 50 on the bone at the planned (or pre-determined) location. The user may then advance a saw blade through the guide slots to form the remaining cut surfaces on the bone in the planned locations. The implant 12 is then mounted on the bone to complete the procedure. 100721 It should be appreciated that while guides (70a-70f) are termed “drill guides” herein, any tool, implant, or implement may be guided through the bores (76, 78, 94a, 94b) to form features, or insert implants, into a bone at a pre-defined distance apart. Thus, the term “drill guide” should not be construed to limit the guide for use only with a drill and is merely used as an example that a drill may be but one device that can be used for forming features, or inserting implants, in the bone with the use of the guide (70a-70f).

### Computer-Assisted Surgical System

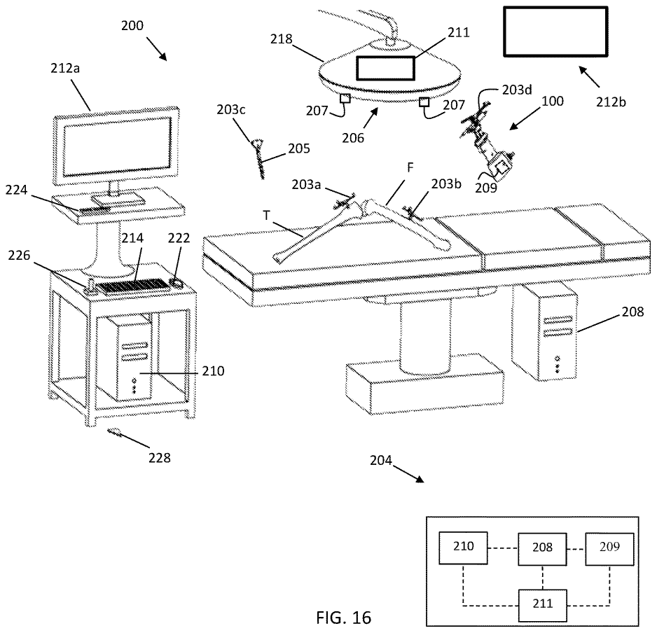

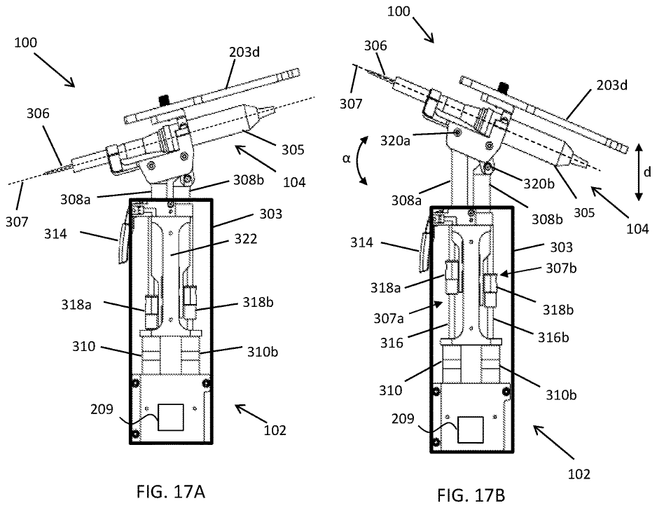

Referring now to FIGS. 16, 17A, and 17B, embodiments of the present inventive system and method generally includes a computer-assisted surgical system. In some inventive embodiments, a hand-held robot 100 is provided for maintaining alignment of an axis of an endeffector 206 (e.g., pin, drill bit, implant) coincident with a virtual plane. FIG. 16 is a schematic view showing the computer-assisted surgical system 200 including a hand-held robot 100, a computing system 204, and a tracking system 206.

The computing system 204 generally includes hardware and software for executing a surgical procedure. By way of example but not limitation, in one preferred form of the present invention, the computing system 204 is configured to control the actuation of the working portion 104 relative to the hand-held portion 102 of the hand-held robot 100 to maintain alignment of the end-effector axis 307 (FIG. 17A) coincident with a virtual plane having a predefined location with respect to a bone. The end-effector 206 coupled to the working portion 104 in operation modifies (e.g., inserts pins, cuts, mills, etc.) subject bone. The computing system 204 may generate control signals to accurately maintain the end-effector axis 207 coincident with a virtual plane based on: a) the location of the virtual plane as registered to the location of the bone (or more specifically to the coordinate system of a tracking array affixed to the bone); and b) the tracked POSE of the hand-held robot 100.

The computing system 204 of the computer-assisted surgical system 200 may include: one or more device computers (208, 209) including a planning computer 210; a tracking computer 211, and peripheral devices. Each computer may include one or more processors. Processors operate in the computing system 204 to perform computations and execute software associated with the inventive system and method. The device computer(s) (208, 209), the planning computer 210, and the tracking computer 211 may be separate entities as shown in FIG. 16, or it is also contemplated that operations may be executed on one (or more) computers depending on the configuration of the computer-assisted surgical system 200. For example, the tracking computer 211 may have operational data to control the hand-held robot 100 without the need for a device computer (208, 209). Furthermore, if desired, any combination of the device computers (208, 209), planning computer 210, and/or tracking computer 211 may be connected together via a wired or wireless connection. It is further appreciated that one or more of the computers may be readily located remote from the surgical site. Cloud-based computation is also contemplated in the present invention. In addition, the data gathered by, and/or the operations performed by, the tracking computer 211 and device computer(s) (208, 209) may work together to control the hand-held robot 100 and, as such, the data gathered by, and/or the operations performed by, the tracking computer 211 and device computer(s) (208, 209) to control the handheld robot 100 may be referred to herein as a “control system”.

The peripheral devices allow a user to interface with the computing system 204 and may include, but are not limited to, one or more of the following: one or more user-interfaces, such as a display or monitor (212a, 212b) to display a graphical user interface (GUI); and userinput mechanisms, such as a keyboard 214, mouse 222, pendent 224, joystick 226, and foot pedal 228. If desired, the monitor(s) (212a, 212b) may have touchscreen capabilities, and/or the handheld robot 100 may include one or more input mechanisms (e.g., buttons, switches, etc.). Another peripheral device may include a tracked digitizer probe 205 to assist in the registration process, or to digitize other objects or locations located in the surgical environment. A tracking array 203 is coupled to the digitizer probe 205 to permit the tracking system 206 to track the POSE of the digitizer probe 205 in space. The digitizer probe 205 may further include one or more user input mechanisms to provide input to the computing system 204. For example, a button on the digitizer probe 205 may allow the user to signal to the computing system 204 to digitize a point in space to assist in registering a bone to a surgical plan.

The device computer(s) (208, 209) may include one or more processors, controllers, software, data, utilities, and/or storage medium(s) such as RAM ROM or other non-volatile or volatile memory to perform functions related to the operation of the hand-held robot 100. By way of example but not limitation, one or more of the device computers (208, 209) may include software to control the hand-held robot 202, e.g., generate control signals for the actuators to move the working portion 104 relative to the hand-held portion 102 to a targeted POSE, receive and process tracking data, control the rotational or oscillating speed of the end-effector 306 by controlling motor 305, execute registration algorithms, execute calibration routines, provide workflow instructions to the user throughout a medical procedure, as well as any other suitable software, data or utilities required to successfully perform the procedure in accordance with embodiments of the invention.

In some embodiments, the system 200 may include a first device computer 208 located separate from the hand-held robot 100 and a second device computer 209 housed in the hand-held robot 100 to provide on-board control. The first device computer 208 may be dedicated to the control of the surgical workflow via a GUI, the registration process and the associated calculations, the display of 3-D models and 3-D model manipulation or animation, performing calculations related to balancing the knee during TKA (e.g., gap balancing), as well as other processes. The second device computer 209, also referred to herein as an on-board device computer, may be dedicated to the control of the hand-held robot 100. For example, the on-board device computer 209 may compute and generate the control signals for the actuator motors (210a, 210b) based on: i) received signals/data corresponding to the real-time POSE of the hand-held robot 100 from the tracking system (e.g., POSE data of a tracking array (or fiducials) coupled to the hand-held robot 100); and ii) received signals/data corresponding to the real-time POSE of the virtual plane (e.g., POSE data of a tracking array (or fiducials) affixed to a bone and, in some embodiments, registration data correlating the POSE data to the predetermined location of the virtual plane). The on-board device computer 209 may also send internal data (e.g., operational data, actuator/screw position data, battery life, etc.) via a wired or wireless connection. In some inventive embodiments, wireless optical communication is used to send and receive the signals/data described herein. Details about bi-directional optical communication between a hand-held robot 100 and a tracking system 206 are further described below.

The planning computer 210 in some inventive embodiments is dedicated to planning the procedure. By way of example but not limitation, the planning computer 210 may contain hardware (e.g., processors, controllers, memory, etc.), planning software, data, and/or utilities capable of: receiving, reading, and/or manipulating medical imaging data; segmenting imaging data; constructing and manipulating three-dimensional (3D) virtual bone models; storing and providing computer-aided design (CAD) files such as implant CAD files; planning the POSE for cut surfaces, device operating data (e.g., virtual planes), screws, pins, implants, grafts, and fixation hardware relative to pre-operative bone data; generating the surgical planning data for use with the system 200, and providing other various functions to aid a user in planning the surgical procedure. The final surgical plan data may include one or more images of a bone or virtual models of the bone, registration data, subject identification information, the POSE for inserting or mounting one or more pins, screws, implants, grafts, fixation hardware relative to the bone, and/or the POSE of device operating data (e.g., virtual planes) defined relative to the bone models. The device computer(s) (208, 209) and the planning computer 210 may be directly connected in the operating room, or the planning computer 210 may exist as separate entities outside the operating room. The final surgical plan is readily transferred to a device computer (208, 209) and/or tracking computer 211 through a wired (e.g., electrical connection) or a wireless connection (e.g., optical communication) in the operating room; or transferred via a non-transient data storage medium (e.g., a compact disc (CD), or a portable universal serial bus (USB drive)) if the planning computer 210 is located outside the operating room (or if otherwise desired). As described above, the computing system 204 may comprise one or more computers, with multiple processors capable of performing the functions of the device computer 208, the tracking computer 211, the planning computer 210, or any combination thereof.

The tracking system 206 of the present invention generally includes a detection device to determine the POSE of an object relative to the position of the detection device. In particular embodiments of the present invention, the tracking system 206 is an optical tracking system such as the optical tracking system described in U.S. Pat. No. 6,061,644 (which patent is hereby incorporated herein by reference), having two or more optical detectors 207 (e.g., cameras) for detecting the position of fiducial markers arranged on rigid bodies or integrated directly on the tracked object. By way of example but not limitation, the fiducial markers may include an active transmitter, such as an LED or electromagnetic radiation emitter; a passive reflector, such as a plastic sphere with a retro-reflective film; or a distinct pattern or sequence of shapes, lines or other characters. A set of fiducial markers arranged on a rigid body, or integrated on a device, is sometimes referred to herein as a tracking array (203a, 203b, 203c, 203d), where each tracking array has a unique geometry/arrangement of fiducial markers, or a unique transmitting wavelength/frequency (if the markers are active LEDS), such that the tracking system 206 can distinguish between each of the tracked objects.

If desired, the tracking system 206 may be incorporated into an operating room light 218, located on a boom, a stand, or built into the walls or ceilings of the operating room. The tracking system computer 211 includes tracking hardware, software, data, and/or utilities to determine the POSE of objects (e.g., tissue structures, the hand-held robot 100) in a local or global coordinate frame. The output from the tracking system 206 (i.e., the POSE of the objects in 3-D space) is referred to herein as tracking data, where this tracking data may be readily communicated to the device computer(s) (208, 209) through a wired or wireless connection. In a particular embodiment, the tracking computer 206 processes the tracking data and provides control signals directly to the hand-held robot 100 and/or device computer 208 based on the processed tracking data to control the position of the working portion 104 of the hand-held robot 100 relative to the hand-held portion 102. In another embodiment, the tracking computer 206 sends tracking data to a receiver located on the hand-held robot 100, where an on-board device computer 209 generates control signals based on the received tracking data.

The tracking data is determined in some inventive embodiments using the position of the fiducial markers detected from the optical detectors and operations/processes such as image processing, image filtering, triangulation algorithms, geometric relationship processing, registration algorithms, calibration algorithms, and coordinate transformation processing. These operations/processes may be executed directly on the tracking system computer 211 or executed on a separate computer (e.g., first device computer 208) in communication with the tracking system 206.

Bi-directional optical communication (e.g., light fidelity or Li-Fi) may occur between the hand-held robot 100 and the tracking system 206 by way of a modulated light source (e.g., light emitting diode (LED)) and a photosensor (e.g., photodiode, camera). The hand-held robot 100 may include an LED and a photosensor (i.e., a receiver) disposed on the working portion 104 or hand-held portion 102, where the LED and photosensor are in communication with a processor such as modem or an on-board device computer. Data generated internally by the hand-held robot 100 may be sent to the tracking system 206 by modulating the LED, where the light signals (e.g., infrared, visible light) created by the modulation of the LED are detected by the tracking system optical detectors (e.g., cameras) or a dedicated photosensor and processed by the tracking system computer 211. The tracking system 206 may likewise send data to the hand-held robot 100 with a modulated LED associated with the tracking system 206. Data generated by the tracking system 206 may be sent to the hand-held robot 202 by modulating the LED on the tracking system 206, where the light signals are detected by the photosensor on the hand-held robot 100 and processed by a processor in the hand-held robot 100. Examples of data sent from the tracking system 206 to the hand-held robot 100 includes operational data, surgical planning data, informational data, control data, positional or tracking data, pre-operative bone data, or instructional data. Examples of data sent from the hand-held robot 100 to the tracking system 206 may include motor position data, battery life, operating status, logged data, operating parameters, warnings, or faults. In some embodiments, data generated by the first device computer 208 is sent to the tracking system 206, where that generated data is transferred to the hand-held robot 100 via the LED on the tracking system 206.

It should be appreciated that in some embodiments of the present invention, other tracking systems are incorporated with the surgical system 200. By way of example but not limitation, the surgical system 200 may comprise an electromagnetic field tracking system, ultrasound tracking systems, accelerometers and gyroscopes, and/or a mechanical tracking system. The replacement of a non-mechanical tracking system with other tracking systems will be apparent to one skilled in the art in view of the present disclosure. In one form of the present invention, the use of a mechanical tracking system may be advantageous depending on the type of surgical system used such as the computer-assisted surgical system described in U.S. Pat. No. 6,322,567 assigned to the assignee of the present application and incorporated herein by reference in its entirety.

FIGS. 17A and 17B are schematic views showing the hand-held robot 100 in greater detail. More particularly, FIG. 17A shows the hand-held robot 100 in a first working POSE, and FIG. 17B illustrates the hand-held robot 100 in a second working POSE. The hand-held robot 100 comprises a hand-held portion 102 (or handle) and a working portion 104. The hand-held portion 102 comprises an outer casing 303 of ergonomic design which can be held and wielded by a user (e.g., a surgeon). In particular embodiments, the hand-held robot 100 is intended to be fully supported by the hands of the user in that there are no additional supporting links connected to the hand-held robot 100 and the user supports the full weight of the hand-held robot 100. The working portion 104 comprises an end-effector 306 (e.g., pin, drill bit) having an end-effector axis 307. The end-effector 306 may be removably coupled to the working portion 104 (via a coupler (e.g., chuck)) and driven by a motor 305. The hand-held portion 102 and working portion 104 are connected to one another, for example, by a first linear actuator 307a and a second linear actuator 307b in order to control the pitch and translation of the working portion 104 relative to the hand-held portion 102, as will hereinafter be discussed in further detail. In this configuration, the hand-held robot 100 is a 2 degree-of-freedom robot capable of controlling movement of the working portion 104 relative to the hand-held portion 102 in 2 degrees-offreedom (pitch and translation). In a particular embodiment, the working portion 104 is removably coupled to the hand-held portion 102 to permit different types of working portions to be assembled to the hand-held portion 102. For example, a first working portion 104 may illustratively be a laser system having components to operate a laser for treating tissue, a second working portion 104 may illustratively be a drill for rotating a bone pin, and a third working portion 104 may illustratively be an oscillating saw.

A tracking array 203d, having three or more fiducial markers of the sort well known in the art, is preferably rigidly attached to the working portion 104 in order to permit the tracking system 206 (FIG. 10) to track the POSE of the working portion 104. The three or more fiducial markers may, alternatively, be integrated directly with the working portion 104. The fiducial markers may be active markers such as light emitting diodes (LEDs), or passive markers such as retroreflective spheres. The hand-held robot 100 may further include one or more user input mechanisms such as triggers (e.g., trigger 314) or button(s). The user input mechanisms may permit the user to perform various functions illustratively including: activating or deactivating the motor 305; activating or deactivating the actuation of the working portion 104 relative to the hand-held portion 102; notifying the computing system 204 to change from targeting one virtual plane to a subsequent virtual plane; and pausing the surgical procedure.

Within the outer casing of the hand-held portion 102 is the a linear actuator 307a and a second linear actuator 307b. Each linear actuator (307a, 307b) may include a motor (3 IOa, 310b) to power a screw (316a, 316b) (e.g., a lead screw, a ball screw), a nut (318a, 318b), and a linear member (308a, 308b). In some inventive embodiments, the motors (first motor 310a, second motor 310b) are electric servo-motors that bi-directionally rotate the screws (316a, 316b). Motors (310a, 310b) may also be referred to herein as linear actuator motors. The nuts (318a, 318b) (e.g., ball nuts, elongated nuts) are operatively coupled to the screws (316a, 316b) to translate along the screws (316a, 316b) as each screw is rotated by its respective motor (3 IOa, 310b). A first end of each linear member (308a, 308b) is coupled to a corresponding nut (316a, 316b) and the opposing end of each linear member (308a, 308b) is coupled to the working portion 104 via hinges/links (320a, 320b), such that the hinges/links (320a, 320b) allow the working portion 104 to pivot relative to the linear rails (308a, 308b). The motors (3 IOa, 310b) power the screws (316a, 316b) which in turn cause the nuts (318a, 318b) to translate along the axis of the screws (316a, 316b). Translation of nuts 318a, 318b along ball screws 316a, 316b, respectively, causes translation of front linear member 308a and back linear member 308b, respectively, whereby to permit (a) selective linear movement of working portion 104 relative to hand-held portion 102, and (b) selective pivoting of working portion 304 relative to hand-held portion 302 of hand-held robot 100. Accordingly, the translation “d” and pitch “u” (FIG. 17B) of the working portion 104 may be adjusted depending on the position of each nut (318a, 318b) on their corresponding screw (316a, 316b). A linear guide 322 (FIG. 17A) may further constrain and guide the motion of the linear member (308a, 308b) in the translational direction “d”. In a particular embodiment, the nuts (316a, 316b) are elongated and couple directly to the working portion 104 via the hinges/links (320a, 320b), in which case the linear member (308a, 308b) are no longer a component of the linear actuators (307a, 307b). It should be appreciated that other linear actuation mechanisms/components may be used to adjust the POSE of the working portion 104 relative to the hand-held portion 102 such as linear motors, pneumatic motors, worm drives and gears, rack and pinion gears, and other arrangements of motors and transmissions.

The hand-held robot 100 may receive power via an input/output port (e.g., from an external power source) and/or from on-board batteries (not shown).

The motors (305, 310a, 310b) of the hand-held robot 100 may be controlled using a variety of methods. By way of example but not limitation, according to one method of the present invention, control signals may be provided via an electrical connection to an input/output port. By way of further example but not limitation, according to another method of the present invention, control signals are communicated to the hand-held robot 100 via a wireless connection, thereby eliminating the need for electrical wiring. The wireless connection may be made via optical communication. In certain inventive embodiments, the hand-held robot 100 includes a receiver for receiving control signals from the computing system 204 (FIG. 10). The receiver may be, for example, an input port for a wired connection (e.g., Ethernet port, serial port), a transmitter, a modem, a wireless receiver (e.g., Wi-Fi receiver, Bluetooth@ receiver, a radiofrequency receiver, an optical receiver (e.g., photosensor, photodiode, camera)), or a combination thereof. The receiver may send control signals from the computing system 204 directly to the motors (305, 310a, 310b) of the hand-held robot 100, or the receiver may be in communication with a computer (e.g., an on-board device computer 209) that processes signals received by the receiver and then generates the control signals for the motors (305, 310a, 310b) based on the received signals.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A guide comprising:
a first guide tube having a first bore sized to accommodate a first bone pin and a bone contacting surface configured to contact a bone surface;
a second guide tube having a second bore sized to accommodate a second bone pin or a surgical boring tool; and
a cross-bar portion interconnecting the first guide tube and the second guide tube, the cross-bar configured to space the first bore a pre-determined distance from the second bore.

2. The guide of claim 1 wherein the first bore and the second bore have the same diameter.

3. The guide of claim 2 wherein the first guide tube is co-linear with the second guide tube.

4. The guide of claim 1 further comprising a vent in communication between the first bore and a first outer wall of the first guide tube or and the second bore and a second outer wall of the second guide tube.

5. The guide of claim 1 further comprising at least one stabilizing feature.

6. The guide of claim 5 wherein the at least one stabilizing is a bone contacting flange, a handle, an aperture in the cross portion, a bone clamp, or combinations thereof.

7. The guide of claim 1 further comprising an opening within the cross-bar portion positioned between the first guide tube and the second guide tube and sized to accommodate a third bone pin.

8. A process for aligning a cut guide on a bone at a pre-determined location using the guide of claim l, comprising:
maintaining alignment of an axis of a first pin coincident with a virtual plane having a pre-defined location with respect to the bone for inserting the first pin in the bone coincident with the virtual plane; and
maintaining alignment of an axis of a second pin coincident with the virtual plane for inserting the second pin in the bone coincident with the virtual plane, wherein the alignment of the axis of the second pin is maintained with the virtual plane while at least a portion of the first pin inserted in the bone is positioned inside the first bore and at least a portion of the second pin is positioned inside the second bore for inserting the second pin in the bone at a pre-defined distance from the first pin, and wherein the first pin and the second pin inserted in the bone each form a hole in the bone for receiving a portion of a cut guide therein for aligning the cut guide on the bone at a pre-determined location.

9. The process of claim 8 wherein the cut guide is a N-in-l cut block.

10. The process of claim 9 wherein the N-in-l cut block is a 4-in-l cut block.

11. The process of claim 8 wherein each of the first pin and the second pin has a diameter corresponding to a diameter of a peg of a N-in-l cut block such that the each hole formed by the first pin and the second pin are configured to receive each peg of the .N-in-l cut block.

12. A process for aligning a cut guide on a bone at a pre-determined location using the guide of claim 7, comprising:
maintaining alignment of an axis of the third bone pin coincident with a virtual plane having a predefined location with respect to the bone for inserting the third bone pin in a cut surface formed on the bone coincident with the virtual plane; and
inserting the third bone pin into the cut surface at an angle relative to the cut surface and coincident with the virtual plane, wherein the third bone pin inserted in the bone is configured to couple with the opening of the guide to align the guide on the bone at the pre-determined location, and wherein the angling of the pin prevents rotation of the guide about the third bone pin.

13. A system, comprising:
the guide of claim l;
a robotic device configured to removably couple to a first tool and a second tool and control movement of the first tool and the second tool; and
a computing system comprising one or more processors configured to:
generate first control signals for the robotic device to maintain alignment of an axis of a first tool, when coupled to the robotic device, coincident with a virtual plane having a pre-defined location with respect to the bone for inserting the first pin in the bone coincident with the virtual plane; and generate second control signals for the robotic device to maintain alignment of an axis of a second tool, when coupled to the robotic device, coincident with the virtual plane for inserting the second pin in the bone coincident with the virtual plane, wherein the alignment of the axis of the second tool is maintained with the virtual plane while at least a portion of the first tool inserted in the bone is positioned inside the first bore and at least a portion of the second tool is positioned inside the second bore for inserting the second tool in the bone at a pre-defined distance from the first tool inserted in the bone, and wherein the first tool and the second tool inserted in the bone each form a hole in the bone for receiving a portion of a cut guide therein for aligning the cut guide on the bone at a pre-determined location.

14. The system of claim 13 wherein the robotic device is a hand-held robot.

15. The system of claim 14 wherein the hand-held robot comprises:
a hand-held portion;
a working portion movably coupled to the hand-held portion and configured to couple with the first tool and the second tool; and an actuator system configured to move the working portion with respect to the hand-held portion in response to control signals to maintain alignment of the axis of the first tool or the axis of the second tool, when coupled to the working portion, with the virtual plane.

16. The system of claim 15 wherein the first tool is a first pin and the second tool is a boring tool or a second pin.

17. The system of claim 13 further comprising a tracking system for determining a position of the robotic device and a position of the bone.

18. The system of claim 17 wherein the tracking system is an optical tracking system.

19. The system of claim 13 wherein the pre-determined distance between the first bore and the second bore corresponds to a distance between a pair of pegs of a N-in-l cut block.

20. The system of claim 13 wherein the actuator system moves the working portion relative to the hand-held portion in no more than two degrees-of-freedom.
